(19) United States
(12) Patent Application Publication
(10) Pub. No.: US 2026/0047956 A1
(43) Pub. Date: Feb. 19, 2026

(54) FLUID COLLECTION DEVICES INCLUDING A BASE SECUREMENT AREA, AND RELATED SYSTEMS AND METHODS

(71) Applicant: PUREWICK CORPORATION, Covington, GA (US)

(72) Inventors: Ashley Johannes, Atlanta, GA (US); Hollie Trullenque, Houston, TX (US); Eric Rehm, Lawrenceville, GA (US); Michelle Root, Malvern, PA (US)

(21) Appl. No.: 19/310,118

(22) Filed: Aug. 26, 2025

Related U.S. Application Data
(63) Continuation of application No. 17/596,629, now Pat. No. 12,419,778.
(60) Provisional application No. 62/864,656, filed on Jun. 21, 2019.

(51) Int. Cl. A61F 5/455; A61F 5/44; B29D 22/00; B29K 21/00; B29K 83/00

(52) U.S. Cl. CPC A61F 5/455; A61F 5/4404; B29D 22/003; B29K 2021/003; B29K 2083/00

(57) ABSTRACT

Example fluid impermeable barriers of fluid collection devices and methods of forming a fluid impermeable barrier of a fluid collection device. The fluid impermeable barrier includes an inner surface at least partially defining a chamber, a first end region defining an aperture extending therethrough and including a first outer surface portion, and a second end region distal to the first end region and including a second outer surface portion having more adhesive strength than the first outer surface portion. The fluid impermeable barrier also defines an opening extending longitudinally along the fluid impermeable barrier and configured to be positioned adjacent to a female urethra.

### CROSS-REFERENCE TO RELATED APPLICATIONS

This application is a continuation of U.S. patent application Ser. No. 17/596,629 filed on Dec. 15, 2021, which is a U.S. Nationalization of PCT International Application No. PCT/US2020/033064, filed on May 15, 2020, which claims priority to U.S. Provisional Application No. 62/864,656 filed on 21 Jun. 2019, the disclosures of which are incorporated herein, in their entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, pressure ulcers spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are fluid collection devices and methods of assembling fluid collection devices. In an embodiment, a fluid collection device includes a fluid impermeable barrier and a fluid permeable body. The fluid impermeable barrier has an inner surface at least partially defining a chamber, a first end region defining an aperture extending therethrough and including a first outer surface portion, and a second end region distal to the first end region and including a second outer surface portion having more adhesive strength than the first outer surface portion. The fluid impermeable barrier also defines an opening extending longitudinally along the fluid impermeable barrier and configured to be positioned adjacent to a female urethra. The fluid permeable body is positioned at least partially within the chamber to extend across at least a portion of the opening and configured to wick fluid away from the opening.

In an embodiment, a fluid collection device includes a fluid impermeable barrier and a fluid permeable body. The fluid impermeable barrier at least partially defines a chamber, an opening extending longitudinally along the fluid impermeable barrier and configured to be positioned adjacent to a female urethra, and an aperture extending therethrough. At least a portion of the fluid impermeable barrier has a composition including oil and at least one of silicone or thermoplastic elastomer (TPE), the composition including about 60 oil parts per hundred rubber (phr) to about 200 oil phr based on 100 phr of styrene block co-polymer. The fluid permeable body is positioned at least partially within the chamber to extend across at least a portion of the opening and configured to wick fluid away from the opening.

In an embodiment, a method of forming a fluid impermeable barrier of a fluid collection device includes inserting a composition including at least one of silicone or TPE into a barrier mold. The barrier mold includes a first mold portion having a first mold surface and a second portion having a second mold surface more polished than the first mold surface. The method also includes molding the composition in the barrier mold to form a fluid impermeable barrier having an inner surface at least partially defining a chamber, a first end region having a first outer surface portion interfacing the first mold surface, a second end region distal to the first end region and having a second outer surface portion interfacing the second mold surface, and an opening extending longitudinally along the fluid impermeable barrier. The opening is configured to be positioned adjacent to a female urethra, and the second outer surface portion has more adhesive strength than the first outer surface portion.

In an embodiment, a method of forming a fluid impermeable barrier of a fluid collection device includes inserting a composition into at least a portion of a barrier mold, the composition including at least one of silicone or TPE and about 60 oil phr to about 200 oil phr based on 100 phr of styrene block co-polymer. The method also includes molding the composition in the barrier mold to form at least a portion of a fluid impermeable barrier defining a chamber and an opening extending therethrough, the opening configured to be positioned adjacent to a female urethra. The method also includes removing the fluid impermeable barrier from the barrier mold.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a female fluid collection device, according to an embodiment.

FIG. 1B is a front view of the female fluid collection device of FIG. 1A worn on a female user.

FIG. 1C is an exploded view of the female fluid collection device of FIG. 1A.

FIGS. 2A and 2B are cross-sectional views of the female fluid collection device of FIG. 1A taken along line 2-2 thereof, according to various embodiments.

FIG. 3A is an isometric front view of a female fluid collection device, according to a second embodiment.

FIG. 3B is an exploded view of the female fluid collection device of FIG. 3A.

FIG. 3C is a rear isometric view of the female collection device of FIG. 3A.

FIG. 4 is a flow diagram of a method of forming a fluid impermeable barrier of a fluid collection device.

FIG. 5 is a flow diagram of a method of assembling a fluid collection device, according to an embodiment.

FIG. 6 is a flow diagram of a method to collect fluid, according to an embodiment.

FIG. 7 is a block diagram of a system for fluid collection, according to an embodiment.

### DETAILED DESCRIPTION

Embodiments disclosed herein include fluid collection devices and methods of forming fluid collection devices. In female users, fluid collection devices perform better when a portion of the fluid impermeable barrier is positioned at least partially within the gluteal cleft of the wearer. The fluid impermeable barrier of conventional fluid collection devices, however, may slip from between the gluteal cleft, thus rendering the fluid collection device less effective. One or more embodiments of fluid collection devices described herein include a base securement area having an improved outer surface that promote improved adhesion between at least a portion of the fluid impermeable barrier and the gluteal cleft. One or more embodiments of fluid collection devices described herein also include fluid impermeable barriers having a longer longitudinal length, further promoting adhesion of the fluid collection device at least partially within the gluteal cleft of larger or obese users. Exemplary fluid collection devices for use with the systems and methods herein are described in more detail below.

In an embodiment, a fluid collection device includes a fluid impermeable barrier and a fluid permeable body. The fluid impermeable barrier has an inner surface at least partially defining a chamber, a first end region defining an aperture extending therethrough and including a first outer surface portion, and a second end region distal to the first end region and including a second outer surface portion. The second outer surface portion has at least one of more adhesive strength, lower surface roughness, or more tack than the first outer surface portion. The second outer surface portion, then, is more likely to remain secured in the gluteal cleft than conventional fluid collection devices. The fluid impermeable barrier also defines an opening extending longitudinally along the fluid impermeable barrier and configured to be positioned adjacent to a female urethra. The fluid permeable body is positioned at least partially within the chamber to extend across at least a portion of the opening and configured to wick fluid away from the opening.

In an embodiment, a fluid collection device includes a fluid impermeable barrier and a fluid permeable body. The fluid impermeable barrier at least partially defines a chamber, an opening extending longitudinally along the fluid impermeable barrier and configured to be positioned adjacent to a female urethra, and an aperture extending therethrough. At least a portion of the fluid impermeable barrier has a composition including oil and at least one of silicone and thermoplastic elastomer (TPE), the composition including about 60 oil parts per hundred rubber (phr) to about 200 oil phr based on 100 phr of styrene block co-polymer. Incorporation of the oil and at least one of silicone or TPE into the fluid impermeable barrier results in a softer material having more tack that is more likely to remain secured within the gluteal cleft. The fluid permeable body is positioned at least partially within the chamber to extend across at least a portion of the opening and configured to wick fluid away from the opening.

In an embodiment, a method of forming a fluid impermeable barrier of a fluid collection device includes inserting a composition including at least one of silicone or TPE into a barrier mold. The barrier mold includes a first mold portion having a first mold surface and a second portion having a second mold surface more polished than the first mold surface. The method also includes molding the composition in the barrier mold to form a fluid impermeable barrier having an inner surface at least partially defining a chamber, a first end region having a first outer surface portion interfacing the first mold surface, a second end region distal to the first end region and having a second outer surface portion interfacing the second mold surface, and an opening extending longitudinally along the fluid impermeable barrier. The opening is configured to be positioned adjacent to a female urethra. Because the second mold surface is more polished than the first mold surface, the second outer surface portion is also more highly polished than the first outer surface portion, and thus has at least one of more adhesive strength, lower surface roughness, or more tack than the first outer surface portion. The second outer surface portion, then, is more likely to remain secured in the gluteal cleft than conventional fluid collection devices.

In an embodiment, a method of forming a fluid impermeable barrier of a fluid collection device includes inserting a composition into at least a portion of a barrier mold, the composition including at least one of silicone or TPE and about 60 oil phr to about 200 oil phr based on 100 phr of styrene block co-polymer. The method also includes molding the composition in the barrier mold to form at least a portion of a fluid impermeable barrier defining a chamber and an opening extending therethrough, the opening configured to be positioned adjacent to a female urethra. The method also includes removing the fluid impermeable barrier from the barrier mold.

The fluid collection devices disclosed herein are configured to collect fluids from an individual. The fluids collected by the fluid collection devices may include urine. The fluids collected by the fluid collection devices may also include at least one of vaginal discharge, penile discharge, reproductive fluids, blood, sweat, or other bodily fluids.

Fluid collection devices described herein may be used in fluid collection systems. The fluid collection systems may include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

Fluid collection devices described herein may be shaped and sized to be positioned adjacent to the opening of a female urethra or have a male urethra positioned therethrough (e.g., receive a penis therein). For example, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra or have a male urethra positioned therethrough. The fluid collection device may include a fluid permeable body disposed within the fluid impermeable barrier. The conduit may extend into the fluid collection device at a first end region, through one or more of the fluid impermeable barrier, fluid permeable body to a second end region of the fluid collection device. Exemplary fluid collection devices for use with the systems and methods herein are described in more detail below.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through the fluid collection device via the fluid storage container. Fluid, such as urine, may be drained from the fluid collection device using the portable vacuum source.

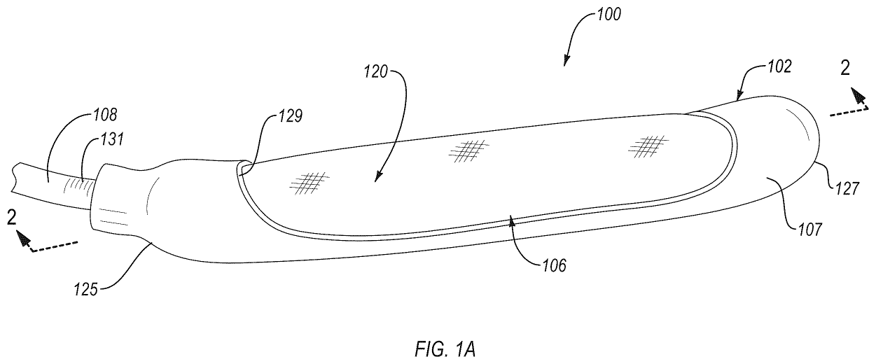

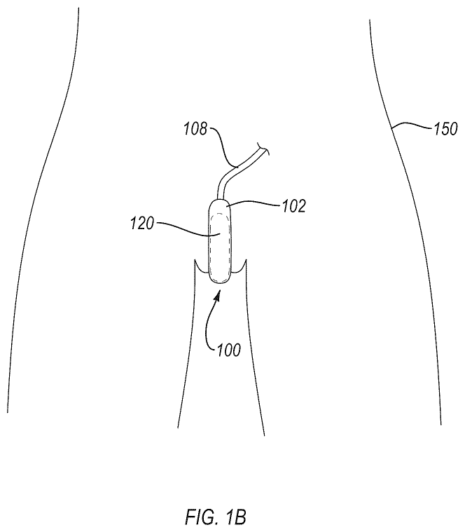

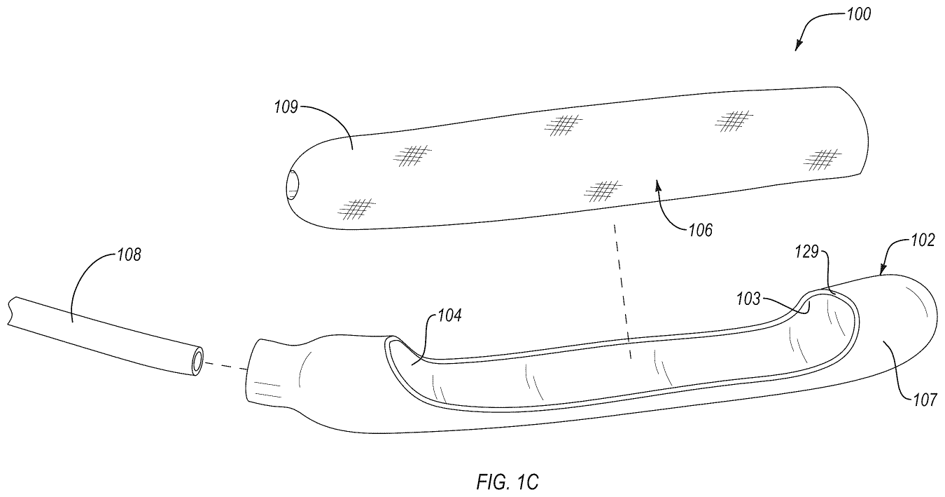

FIG. 1A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one or more of substantially planar surfaces, triangular, or other suitable shapes. The opening 106 is formed in and extends longitudinally through the fluid impermeable barrier 102, thereby enabling fluids to enter the chamber 104 from outside of the fluid collection device 100. The opening 106 may be configured to be positioned adjacent to a female urethra.

The fluid collection device 100 may be positioned proximate to the opening of the female urethra and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening 106 may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arced portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, TPE, oil, rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, allowing the fluid collection device 100 to bend or curve when positioned against the body of a wearer. One or more TPEs may be combined with at least one of silicone and oil. In many embodiments, the fluid impermeable barrier 102 may include a composition having at least silicone and oil therein. A fluid impermeable barrier 102 having oil in the amounts described below provide a fluid impermeable barrier 102 having a softer outer surface 107 that has more adhesive strength or tack than fluid impermeable barriers of conventional fluid collection devices.

For example, a work of adhesion of the fluid impermeable barrier 102 may vary according to different embodiments. The work of adhesion of the fluid impermeable barrier 102 may be about 200 gram seconds (gs) to about 3500 gs, about 200 gs to about 1850 gs, about 1850 gs to about 3500 gs, about 200 gs to about 1025 gs, about 1025 gs to about 1850 gs, about 1850 gs to about 2675 gs, about 2675 gs to about 3500 gs, about 200 gs to about 800 gs, about 600 gs to about 1200 gs, about 1000 gs to about 1600 gs, about 1400 gs to about 2000 gs, about 1800 gs to about 2400 gs, about 2200 gs to about 2800 gs, about 2600 gs to about 3200 gs, about 3000 gs to about 3600 gs, at least about 200 gs, at least about 600 gs, at least about 1000 gs, at least about 1400 gs, at least about 1800 gs, at least about 2200 gs, at least about 2600 gs, at least about 3000 gs, at least about 3400 gs, less than about 3500 gs, less than about 3100 gs, less than about 2700 gs, less than about 2300 gs, less than about 1900 gs, less than about 1500 gs, less than about 1100 gs, less than about 700 gs, or less than about 300 gs.

A tack of the fluid impermeable barrier 102 also may vary according to different embodiments. For example, the tack force of the fluid impermeable barrier 102 may be about 50 grams (g) to about 500 g, about 50 g to about 275 g, about 275 g to about 500 g, about 50 g to about 100 g, about 100 g to about 150 g, about 150 g to about 200 g, about 200 g to about 250 g, about 250 g to about 300 g, about 300 g to about 350 g, about 350 g to about 400 g, about 400 g to about 450 g, about 450 g to about 500 g, at least about 50 g, at least about 100 g, at least about 150 g, at least about 200 g, at least about 250 g, at least about 300 g, at least about 350 g, at least about 400 g, at least about 450 g, less than about 100 g, less than about 150 g, less than about 200 g, less than about 250 g, less than about 300 g, less than about 350 g, less than about 400 g, less than about 450 g, or less than about 500 g.

The type of oil in the fluid impermeable barrier 102 may vary according to different embodiments. In some embodiments, the fluid impermeable barrier 102 may include mineral oil. The amount of oil in the fluid impermeable barrier 102 may vary according to different embodiments. For example, the fluid impermeable barrier 102 may include a composition having varying oil phr based on 100 phr of styrene block co-polymer, such as about 60 oil phr to about 200 oil phr, about 50 oil phr to about 100 oil phr, about 100 oil phr to about 150 oil phr, about 150 oil phr to about 200 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, about 160 oil phr to about 180 oil phr, about 180 oil phr to about 200 oil phr, about 60 oil phr to about 70 oil phr, about 70 oil phr to about 80 oil phr, about 80 oil phr to about 90 oil phr, about 90 oil phr to about 100 oil phr, about 100 oil phr to about 110 oil phr, about 110 oil phr to about 120 oil phr, about 120 oil phr to about 130 oil phr, about 130 oil phr to about 140 oil phr, about 140 oil phr to about 150 oil phr, about 150 oil phr to about 160 oil phr, about 160 oil phr to about 170 oil phr, about 170 oil phr to about 180 oil phr, about 180 oil phr to about 190 oil phr, about 190 oil phr to about 200 oil phr, less than about 200 oil phr, less than about 180 oil phr, less than about 160 oil phr, less than about 140 oil phr, less than about 120 oil phr, less than about 100 oil phr, less than about 80 oil phr, less than about 60 oil phr, greater than about 200 oil phr, greater than about 180 oil phr, greater than about 160 oil phr, greater than about 140 oil phr, greater than about 120 oil phr, greater than about 100 oil phr, greater than about 80 oil phr, or greater than about 60 oil phr. The material of the fluid impermeable barrier 102 may, in some examples, include TPE materials having oil mixed therein, such as Versaflex 2000 or Dynaflex G6713.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material.

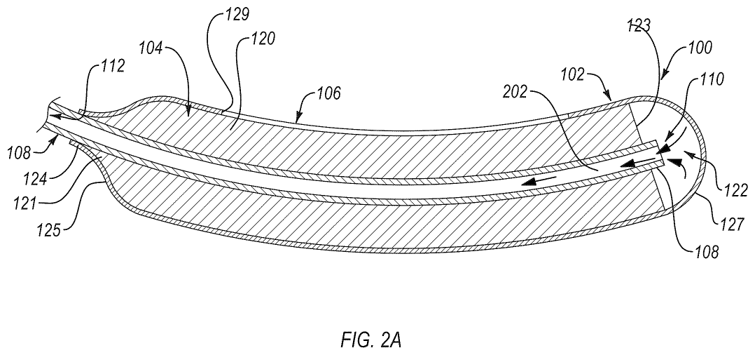

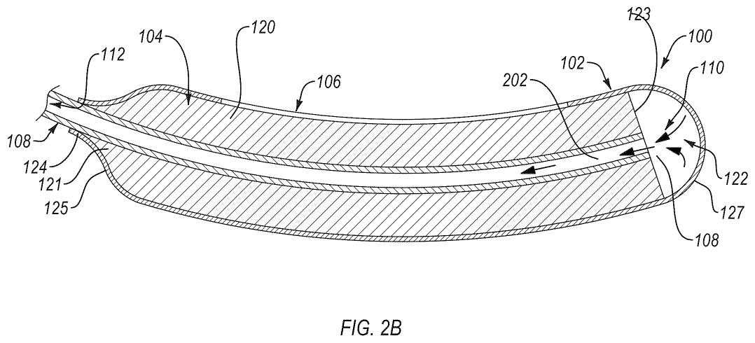

The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIGS. 2A and 2B) of void space formed within the chamber 104. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1C) of the fluid permeable body 120 interfaces with an inner surface 103 (shown in FIG. 1C) of the fluid impermeable barrier 106. A singular fluid permeable body 120 may be advantageous to conventional systems, which typically require an air-laid nonwoven pad covered by a ribbed fabric compression bandage, because a singular fluid permeable body 120 reduced the number of components in the fluid collection device 100, reduces the assembly time of the fluid collection device 100, requires shelf-life data for only a single component, and provides a latex-free single component. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface 109 and a single layer or type of material between the opening 106 and the conduit 108 positioned within the fluid permeable body 120.

In other embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable support, with both the fluid permeable membrane and the fluid permeable support being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating. Such “wicking” may not include absorption into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material.

The fluid permeable membrane may also wick the fluid generally towards an interior of the chamber 104, as discussed in more detail below. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous nylon structure (e.g., spun nylon fibers) and a fluid permeable membrane including gauze about or over the porous nylon structure.

FIG. 1B is a front view of a fluid collection device 100 in use on a female user 150. In use, the fluid permeable body 120 of the fluid collection device is positioned adjacent to a urethra of the user 150. The fluid permeable body 120 is disposed within a chamber 104 (shown in FIGS. 2A and 2B) of the fluid impermeable barrier 102 of the fluid collection device 100 and is exposed to the urethra of the user 150 through the opening 106 in the fluid collection device 100. The fluid collection device 100 may be secured to the user with any of a number of securing devices. Fluids received in the chamber 104 of the fluid collection device 100 from the urethra may be removed through the conduit 108.

FIG. 2A is a cross-sectional view of the fluid collection device 100 taken along line 2-2 of FIG. 1A. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a TPE, polyvinyl chloride, ethylene vinyl acetate, polytetrafluorocthylene, etc., tubing. In some embodiments, the conduit 108 may include silicone or latex.

The fluid impermeable barrier 102 may store fluids in the reservoir 122 thercin. The reservoir 122 is an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 2A, the reservoir 122 is defined by the second body end 123 of the fluid permeable body 120 and the second end region 127 of the fluid impermeable barrier 122. However, the reservoir 122 may be located at the portion of the chamber 104 that is closest to the inlet 110 (e.g., the second end region). The reservoir 122 also may be located at different locations in the chamber 104. For example, the reservoir 122 may be located at the end of the chamber 104 that is closest to the outlet 112. In these and other embodiments, the conduit 108 may extend through the first region 125 of the fluid impermeable barrier 102 to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108.

Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The apertures 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 2A. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, as shown in FIG. 2B, an end of the conduit 108 may be generally flush or coplanar with the second body end 123 of the fluid permeable body 120. In other embodiments, the end of the conduit 108 may be recessed from the second body end 123 of the fluid permeable body 120. The end of the conduit 108 also may be selectively moveable between partially extending into the reservoir 122 (shown in FIG. 2A) and recessed from or flush with the second body end 123 of the fluid permeable body (shown in FIG. 2B).

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as to by having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a TPE polymer.

In any of the embodiments disclosed herein the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein.

In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

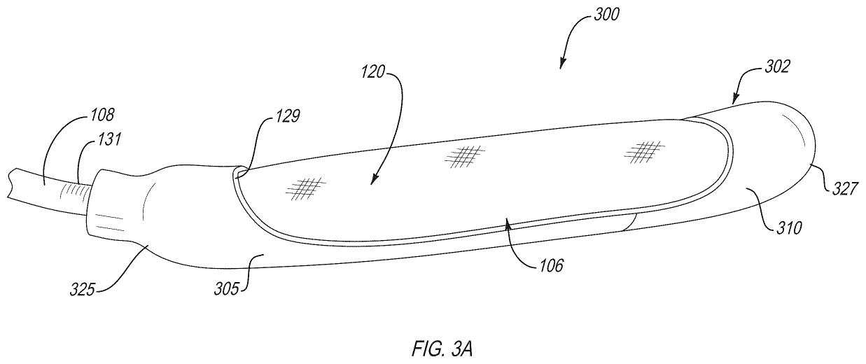

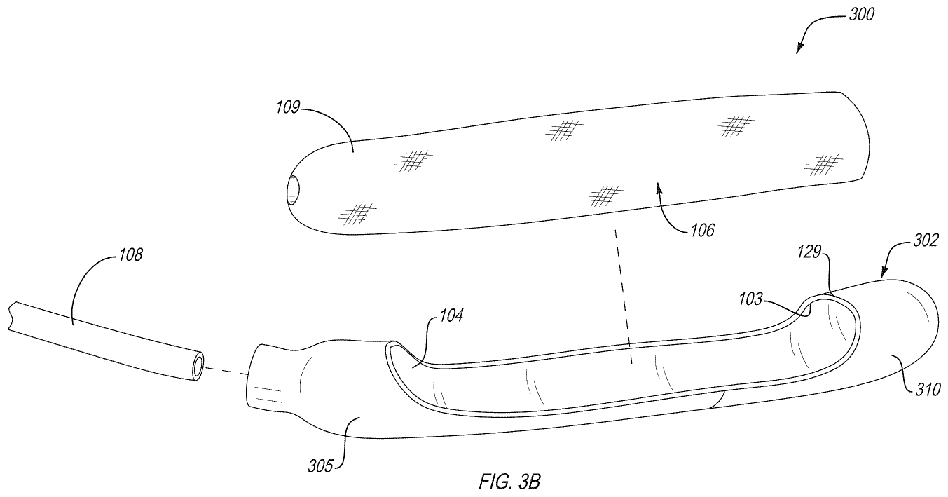

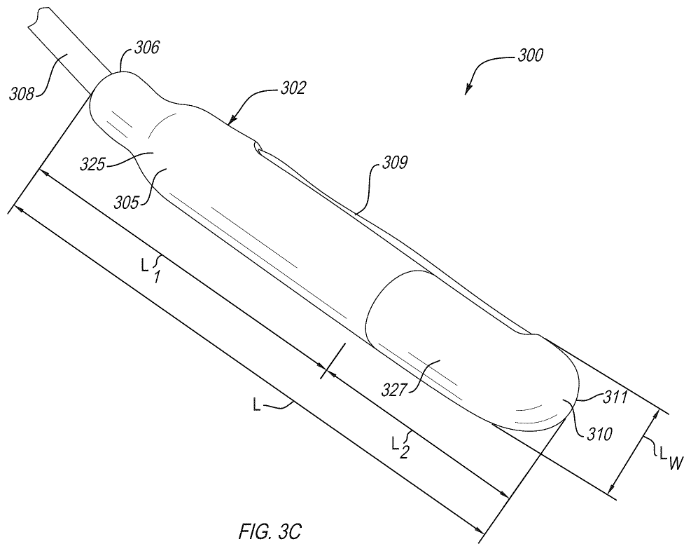

Turning ahead in the drawings to FIG. 3A-C, which provide various views of a fluid collection device 300, according to one or more embodiments. Unless otherwise noted, the fluid collection device 300 may include any of the features described in relation to the fluid collection device 100. Turning to FIG. 3C, the fluid collection device 300 includes a fluid impermeable barrier 302 having a first end region 325 extending from a first end 306 of the fluid impermeable barrier 302 and a second end region 327 extending from a second end 311 of the fluid impermeable barrier 302 towards the first end region 325. The first end region 325 includes a first outer surface portion 305 and the aperture 124, and the second end region includes a second outer surface portion 310.

The fluid impermeable barrier 302 also may include a longitudinal length L extending from the first end 306 to the second end 311. The first outer surface portion 305 may extend a first length L₁ along the longitudinal length L, and the second outer surface portion 310 may extend a second length L₂ along the longitudinal length L. The second length L₂ may vary according to different embodiments. For example, the second length L₂ may be at least about one-tenth the longitudinal length L, at least about one-fifth the longitudinal length L, at least about one-fourth the longitudinal length L, at least about one-third the longitudinal length L, at least about one-half the longitudinal length L, about one-tenth to about one-half the longitudinal length L, about one-fifth to about one-half the longitudinal length L, about one-fourth to about one-half the longitudinal length L, about one-third to about one-half the longitudinal length L, about one-tenth the longitudinal length L, about one-fifth the longitudinal length L, about one-fourth the longitudinal length L, about one-third the longitudinal length L, about one-half the longitudinal length L, less than about one-tenth the longitudinal length L, less than about one-fifth the longitudinal length L, less than about one-fourth the longitudinal length L, less than about one-third the longitudinal length L, or less than about one-half the longitudinal length L.

The longitudinal length L of the fluid impermeable barrier 302 may vary according different embodiments. In some embodiments, the fluid impermeable barrier 302 may include a longer longitudinal length than conventional fluid collection devices. A longer longitudinal length is advantageous to shorter, conventional fluid collection devices because a longer fluid impermeable barrier 302 allows for easier placement and use of the fluid collection device 300 on obese or larger patients. While specific reference is made to the fluid impermeable barrier 302, the fluid impermeable barrier 102 also may include any of the longitudinal lengths and lateral widths described in relation to the fluid impermeable barrier 302. The longitudinal length L of the fluid impermeable barrier 302 may be about 7.5 inches (about 19.0 cm) to about 20 inches (about 50.8 cm), about 7.5 inches to about 15 inches (about 38.1 cm), about 7.5 inches to about 13 inches (about 33.0 cm), about 7.5 inches to about 11 inches (about 27.9 cm), about 7.5 inches to about 9 inches (about 22.9 cm), about 9 inches to about 20 inches, about 9 inches to about 15 inches, about 9 inches to about 13 inches (about 33.0 cm), about 9 inches to about 11 inches, about 10 inches (about 25.4 cm) to about 20 inches, about 10 inches to about 15 inches, about 10 inches to about 13 inches, about 10 inches to about 11 inches, at least about 7.5 inches, at least about 8 inches (about 20.3 cm), at least about 9 inches, at least about 10 inches, at least about 11 inches, at least about 12 inches (about 30.4 cm), at least about 13 inches, at least about 14 inches (about 35.6 cm), at least about 15 inches, at least about 20 inches, about 7.5 inches, about 8 inches, about 9 inches, about 10 inches, about 11 inches, about 12 inches, about 13 inches, about 14 inches, about 15 inches, or about 20 inches.

The fluid impermeable barrier 302 also may include a diameter or lateral width L_{w}. The lateral width L_{w} of the fluid impermeable barrier may be about 0.5 inch (about 1.3 cm) to about 2.0 inches (about 5.1 cm), about 0.5 inch to about 1.5 inches (about 3.8 cm), about 0.5 inch to about 1.25 inches (about 3.18 cm), about 0.5 inches to about 1.0 inch (about 2.5 cm), about 0.5 inch to about 0.75 inch (about 1.9 cm), about 0.75 inch to about 2.0 inches, about 0.75 inch to about 1.5 inches, about 0.75 inch to about 1.25 inches, about 0.75 inches to about 1.0 inch, about 1.0 inch to about 2.0 inches, about 1.0 inch to about 1.5 inches, about 1.0 inch to about 1.25 inches, about 0.5 inch, about 0.75 inch, about 1 inch, about 1.25 inches, about 1.5 inches, about 1.75 inches (about 4.4 cm), about 2.0 inches, less than about 0.5 inch, less than about 0.75 inch, less than about 1 inch, less than about 1.25 inches, less than about 1.5 inches, less than about 1.75 inches, or less than about 2.0 inches.

In many embodiments, the first outer surface portion 305 of the first end region 325 has different properties than the second outer surface portion 310 of the second end region 327. The second outer surface portions 310 may include a base securement area configured to improve securement of the fluid collection device 300 to a user. For example, the second outer surface portion 327 may have a lower surface roughness, more tack, and/or more adhesive strength than the first outer surface portion. In the illustrated embodiment, the transition between the first outer surface portion 305 of the first end region 325 and the second outer surface portion 310 of the second end region 327 is sharp or abrupt. However, in other embodiments, the transition between the first outer surface portion 305 of the first end region 325 and the second outer surface portion 310 of the second end region 327 is gradual and the properties of the outer surface of the fluid impermeable barrier 302 between the first outer surface portion 305 and the second out surface portion 310 may gradually transition or change.

In one or more embodiments, the first end region 325 and the second end region 327 may include different compositions from one another, thereby resulting in the first outer surface portion 305 having different properties than the second outer surface portion 310. For example, a composition of the second end region 327 may include more oil than a composition of the first end region 325, thereby resulting in the second outer surface portion 310 being more soft and/or having more adhesive strength or tack than first outer surface portion 305. The compositions of both the first end region 325 and the second end region 327 also may include other components as described above in relation to the fluid impermeable barrier 102. The amount of oil in the second end region 327 may vary according to different embodiments. For example, second end region 327 may include a composition having varying oil phr based on 100 phr of styrene block co-polymer, such as about 60 oil phr to about 200 oil phr, about 50 oil phr to about 100 oil phr, about 100 oil phr to about 150 oil phr, about 150 oil phr to about 200 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, about 160 oil phr to about 180 oil phr, about 180 oil phr to about 200 oil phr, about 60 oil phr to about 70 oil phr, about 70 oil phr to about 80 oil phr, about 80 oil phr to about 90 oil phr, about 90 oil phr to about 100 oil phr, about 100 oil phr to about 110 oil phr, about 110 oil phr to about 120 oil phr, about 120 oil phr to about 130 oil phr, about 130 oil phr to about 140 oil phr, about 140 oil phr to about 150 oil phr, about 150 oil phr to about 160 oil phr, about 160 oil phr to about 170 oil phr, about 170 oil phr to about 180 oil phr, about 180 oil phr to about 190 oil phr, about 190 oil phr to about 200 oil phr, less than about 200 oil phr, less than about 180 oil phr, less than about 160 oil phr, less than about 140 oil phr, less than about 120 oil phr, less than about 100 oil phr, less than about 80 oil phr, less than about 60 oil phr, greater than about 200 oil phr, greater than about 180 oil phr, greater than about 160 oil phr, greater than about 140 oil phr, greater than about 120 oil phr, greater than about 100 oil phr, greater than about 80 oil phr, or greater than about 60 oil phr.

When the composition of the second end region 327 includes oil, the composition of the first end region 325 may have a lower oil content than the second end region 327. For example, the first end region 325 may include a composition having no oil or having varying oil phr based on 100 phr of styrene block co-polymer, such as about 0 oil phr to about 150 oil phr, about 1 oil phr to about 50 oil phr, about 50 oil phr to about 100 oil phr, about 100 oil phr to about 150 oil phr, about 1 oil phr to about 20 oil phr, about 20 oil phr to about 40 oil phr, about 40 oil phr to about 60 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, less than about 160 oil phr, less than about 140 oil phr, less than about 120 oil phr, less than about 100 oil phr, less than about 80 oil phr, less than about 60 oil phr, less than about 40 oil phr, or less than about 20 oil phr.

In many embodiments, the second end region 327 may be more highly polished than the first end region 325, thereby resulting in the first outer surface portion 305 having different properties than the second outer surface portion 310. The second end region 327 may have lower surface roughness than the first end region 325 due at least in part to formation of the fluid impermeable barrier 302 in a mold having different surface finishes. For example, the fluid impermeable barrier 302 may be molded in a barrier mold having a first mold portion that interfaces that first outer surface portion 305 and a second mold portion that interfaces the second outer surface portion 310, the second mold portion being more polished or having a smoother surface finish than the first mold portion. The resulting second outer surface portion 310 has more adhesive strength, lower surface roughness, and/or greater tack than the first outer surface portion 305.

For example, in some embodiment, the second mold portion may include a surface having a Society of the Plastics Industry (SPI) rating of A1, A2, A3, B1, B2, or B3. The corresponding second outer surface portion 310, then, also may include a SPI rating approximately equal to the surface of the second mold portion, thus resulting in the second outer surface portion 310 having an SPI rating of about A1, A2, A3, B1, B2, or B3. The first mold portion may include a surface having a SPI rating of C1, C2, C3, D1, D2, or D3. The corresponding first outer surface portion 305, then, also may include a SPI rating approximately equal to the surface of the first mold portion, thus resulting in the first outer surface portion 305 having a SPI rating of C1, C2, C3, D1, D2, or D3.

In many embodiments, the second end region 327 may include an adhesive on the second outer surface portion 310, thereby resulting in the first outer surface portion 305 having different properties than the second outer surface portion 310. The adhesive may be applied to the second outer surface portion 310 after the fluid impermeable barrier 302 is formed, and may include any adhesive suitable for use with skin, such as a hydrogel adhesive. The resulting second outer surface portion 310 has more adhesive strength and/or greater tack than the first outer surface portion 305.

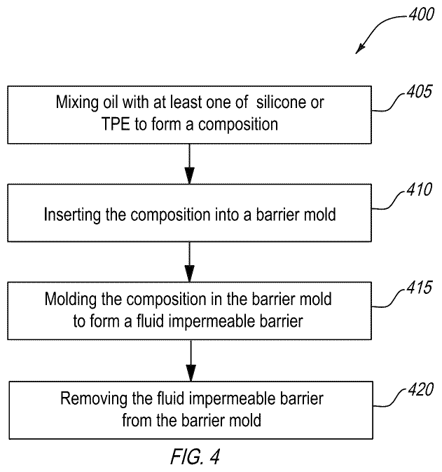

Turning ahead in the drawings to FIG. 4, which is a flow diagram of a method 400 of forming a fluid impermeable barrier of a fluid collection device. The method 400 may optionally include an act 405 of mixing oil with at least one of silicone or TPE to form a composition. The method also includes an act 410 of inserting the composition into a barrier mold. The method also includes an act 415 of molding the composition in the barrier mold to form a fluid impermeable barrier. The method also includes an act 420 of removing the fluid impermeable barrier from the barrier mold

In some embodiments, the method 400 includes the act 405 of mixing oil with at least one of silicone or TPE to form a composition, the composition having varying oil phr based on 100 phr of styrene block co-polymer, such as about 60 oil phr to about 200 oil phr, about 50 oil phr to about 100 oil phr, about 100 oil phr to about 150 oil phr, about 150 oil phr to about 200 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, about 160 oil phr to about 180 oil phr, about 180 oil phr to about 200 oil phr, about 60 oil phr to about 70 oil phr, about 70 oil phr to about 80 oil phr, about 80 oil phr to about 90 oil phr, about 90 oil phr to about 100 oil phr, about 100 oil phr to about 110 oil phr, about 110 oil phr to about 120 oil phr, about 120 oil phr to about 130 oil phr, about 130 oil phr to about 140 oil phr, about 140 oil phr to about 150 oil phr, about 150 oil phr to about 160 oil phr, about 160 oil phr to about 170 oil phr, about 170 oil phr to about 180 oil phr, about 180 oil phr to about 190 oil phr, about 190 oil phr to about 200 oil phr, less than about 200 oil phr, less than about 180 oil phr, less than about 160 oil phr, less than about 140 oil phr, less than about 120 oil phr, less than about 100 oil phr, less than about 80 oil phr, less than about 60 oil phr, greater than about 200 oil phr, greater than about 180 oil phr, greater than about 160 oil phr, greater than about 140 oil phr, greater than about 120 oil phr, greater than about 100 oil phr, greater than about 80 oil phr, or greater than about 60 oil phr.

In some embodiments, the act 405 of mixing oil with at least one of silicone or TPE to form a composition includes mixing one or more constituents to form a first composition and mixing oil with at least one of silicone or TPE to form a second composition having a higher oil content than the first composition. Mixing one or more constituents to form the first composition may include mixing oil with at least one of silicone or TPE to form the first composition, with the first composition having a lower oil content than the second composition. Mixing at least one of silicone or TPE with oil to form the first composition may include mixing oil with at least one of silicone or TPE to form the first composition having varying oil phr based on 100 phr of styrene block co-polymer, such as about 0 oil phr to about 150 oil phr, about 1 oil phr to about 50 oil phr, about 50 oil phr to about 100 oil phr, about 100 oil phr to about 150 oil phr, about 1 oil phr to about 20 oil phr, about 20 oil phr to about 40 oil phr, about 40 oil phr to about 60 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, less than about 160 oil phr, less than about 140 oil phr, less than about 120 oil phr, less than about 100 oil phr, less than about 80 oil phr, less than about 60 oil phr, less than about 40 oil phr, or less than about 20 oil phr

Mixing oil with at least one of silicone or TPE to form the second composition may include mixing oil with at least one of silicone or TPE to form the second composition having varying oil phr based on 100 phr of styrene block co-polymer, such as about 60 oil phr to about 200 oil phr, about 50 oil phr to about 100 oil phr, about 100 oil phr to about 150 oil phr, about 150 oil phr to about 200 oil phr, about 60 oil phr to about 80 oil phr, about 80 oil phr to about 100 oil phr, about 100 oil phr to about 120 oil phr, about 120 oil phr to about 140 oil phr, about 140 oil phr to about 160 oil phr, about 160 oil phr to about 180 oil phr, about 180 oil phr to about 200 oil phr, about 60 oil phr to about 70 oil phr, about 70 oil phr to about 80 oil phr, about 80 oil phr to about 90 oil phr, about 90 oil phr to about 100 oil phr, about 100 oil phr to about 110 oil phr, about 110 oil phr to about 120 oil phr, about 120 oil phr to about 130 oil phr, about 130 oil phr to about 140 oil phr, about 140 oil phr to about 150 oil phr, about 150 oil phr to about 160 oil phr, about 160 oil phr to about 170 oil phr, about 170 oil phr to about 180 oil phr, about 180 oil phr to about 190 oil phr, about 190 oil phr to about 200 oil phr, less than about 200 oil phr, less than about 180 oil phr, less than about 160 oil phr, less than about 140 oil phr, less than about 120 oil phr, less than about 100 oil phr, less than about 80 oil phr, less than about 60 oil phr, greater than about 200 oil phr, greater than about 180 oil phr, greater than about 160 oil phr, greater than about 140 oil phr, greater than about 120 oil phr, greater than about 100 oil phr, greater than about 80 oil phr, or greater than about 60 oil phr

In alternative embodiments of the method 400, a composition including oil and at least one of silicone or TPE may be provided having been previously mixed together. Accordingly, the method 400 may sometimes not include mixing oil with at least one of silicone or TPE. The composition including oil and at least one of silicone or TPE may include, for example, a stock material including oil and at least one of silicone or TPE.

In some embodiments, the act 410 of inserting the composition into the barrier mold may include inserting the composition including at least one of silicone or TPE into the barrier mold. In these and other embodiments, the barrier mold to which the composition including at least one of silicone or TPE is inserted may include a first mold portion having a first mold surface and a second portion having a second mold surface more polished and/or having a lower surface roughness than the first mold surface. For example, the second mold surface may have an SPI rating of A1, A2, A3, B1, B2, or B3, and the first mold surface may have an SPI rating of C1, C2, C3, D1, D2, or D3. In some embodiments, the method 400 also may include inserting the composition into an injection mold for injection molding of the fluid impermeable barrier. In some embodiments, inserting the method 400 also may include inserting the composition into a molding for liquid silicone or TPE rubber molding of the fluid impermeable barrier.

In embodiments of the method 400 including mixing oil with at least one of silicone or TPE to form a composition includes mixing one or more constituents to form a first composition and mixing oil with at least one of silicone or TPE to form a second composition having a higher oil content than the first composition, the act 410 of inserting the composition into the barrier mold also may include inserting the first composition into the first mold portion of the barrier mold and inserting the second composition into the second mold portion of the barrier mold.

In some embodiments, the act 410 of inserting the composition into the barrier mold may include inserting the composition including oil and at least one of silicone or TPE into at least a portion of the of the barrier mold. In these and other embodiments, the act of inserting the composition including oil and at least one of silicone or TPE into at least a portion of the barrier mold also may include inserting an additional composition into an additional portion of the barrier mold. The additional composition may include at least one of silicone or TPE and also have a lower oil content than the composition. In these and other embodiments, inserting the additional composition into the additional portion of the barrier mold includes inserting the additional composition into the additional portion of the barrier mold having a first mold surface, and inserting the composition into at least a portion of the barrier mold includes inserting the composition into the portion of the barrier mold having a second mold surface more polished than the first mold surface.

The act 415 of molding the composition in the barrier mold to form the fluid impermeable barrier may include one or more of heating and cooling the composition in the barrier mold to form the fluid impermeable barrier. In some embodiments, the act 415 of molding the composition in the barrier mold to form a fluid impermeable barrier may include molding the composition in the barrier mold to form at least a portion of a fluid impermeable barrier defining a chamber and an opening extending therethrough, with the opening configured to be positioned adjacent to a female urethra. In embodiments of the method 400 having two or more different compositions formed, the act 415 of molding the composition in the barrier mold may include molding the composition in the barrier mold to form the fluid impermeable barrier having a first end region including the additional or first composition having a lower oil content, and a second end region distal to the first end region and including the second composition or the composition having a higher oil content.

In embodiments of the method 400 in which the barrier mold includes a first mold portion having a first mold surface and a second portion having a second mold surface more polished and/or having a lower surface roughness than the first mold surface, the act 415 of molding the composition in the barrier mold to form a fluid impermeable barrier may include molding the composition in the barrier mold to form a fluid impermeable barrier having an inner surface at least partially defining a chamber, a first end region having a first outer surface portion interfacing the first mold surface, a second end region distal to the first end region and having a second outer surface portion interfacing the second mold surface, and an opening extending longitudinally along the fluid impermeable barrier. In these and other embodiments, the opening is configured to be positioned adjacent to a female urethra, and the second outer surface portion has more adhesive strength than the first outer surface portion.

In some embodiments of the method 400, the method 400 also may include applying an adhesive to the second outer surface portion of the fluid impermeable barrier after the fluid impermeable barrier has been molded. The adhesive applied to the second outer surface portion may include any adhesive suitable for use with skin, such as a hydrogel adhesive. According to an embodiment, a suitable adhesive is a hydrogel layer, such as those disclosed in U.S. Patent Application Publication No. 2017/0189225, the disclosure of which is incorporated herein by reference in its entirety. The resulting second outer surface portion has more adhesive strength and/or greater tack than the first outer surface portion.

The acts 405, 410, 415, and 420 of the method 400 are for illustrative purposes. For example, the acts 405, 410, and 415 of the method 400 may be performed in different orders, split into multiple acts, modified, supplemented, or combined. In an embodiment, one or more of the acts 405, 410, and 415 of the method 400 may be omitted from the method 400. Any of the acts 405, 410, and 415 may include forming any of the fluid impermeable barriers disclosed herein.

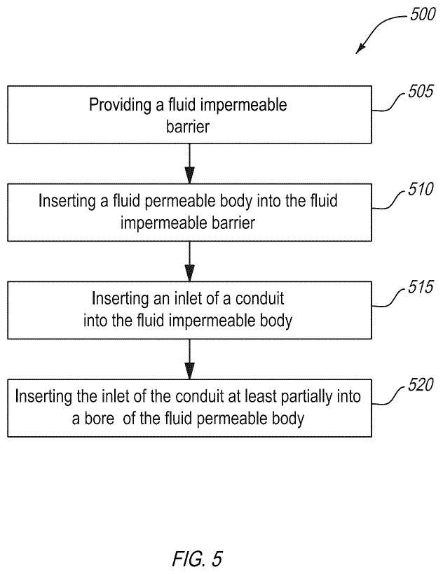

FIG. 5 is a flow diagram of a method 500 of assembling the fluid collection devices and/or fluid collection systems disclosed herein, according to an embodiment. The method 500 may include an act 505, which recites providing a fluid impermeable barrier. The fluid impermeable barrier at least partially defines a chamber and also an opening extending therethrough. The opening is configured to be positioned adjacent to a female urethra or have a male urethra positioned therethrough. The fluid permeable body may include a singular porous hydrophilic polyolefin material extruded, molded, or sintered to a substantially cylindrical shape

The method may include an act 510, which recites inserting a substantially cylindrical and fluid permeable body into the chamber of the fluid impermeable barrier. When the fluid permeable body is inserted into the chamber of the fluid impermeable barrier, the fluid permeable body interfaces at least a portion of the fluid impermeable barrier and covers at least a portion of the opening. The fluid permeable body includes a singular porous material that is substantially cylindrical in shape and configured to wick any fluid away from the opening. In some embodiments, the act 510 may include inserting the fluid permeable body into the chamber of the fluid impermeable barrier such that a reservoir is defined within the chamber by a second body end of the fluid permeable body distal to the first body end and a second end region of the fluid impermeable barrier distal to the aperture. In some embodiments, the act 510 may include inserting the substantially cylindrical and fluid permeable body into the chamber of the fluid impermeable barrier such that the fluid permeable body and the conduit fill substantially all of the chamber.

The method may include an act 515, which recites inserting an inlet of a conduit into the fluid impermeable body. The conduit may be inserted into the fluid impermeable body through an aperture defined by the fluid impermeable barrier at a first end region of the fluid impermeable barrier. In some embodiments, the act 515 may include inserting the inlet of the conduit into the bore at the first body end, through the bore of the fluid permeable body, through the second body end of the fluid permeable body, and into the reservoir such that the conduit extends from the reservoir, through the fluid permeable body, through the aperture to outside the fluid impermeable barrier.

The method may include an act 520, which recites inserting the inlet of the conduit at least partially into a bore at a first body end of the fluid permeable body. The bore extends at least partially through the fluid permeable body and is defined by the fluid permeable body. The conduit interfaces at least a portion of the fluid permeable body.

The acts 505, 510, 515, and 520 of the method 500 are for illustrative purposes. For example, the acts 505, 510, 515, and 520 of the method 500 may be performed in different orders, split into multiple acts, modified, supplemented, or combined. In an embodiment, one or more of the acts 505, 510, 515, and 520 of the method 500 may be omitted from the method 500. Any of the acts 505, 510, 515, and 520 may include using any of the fluid collection devices or systems disclosed herein.

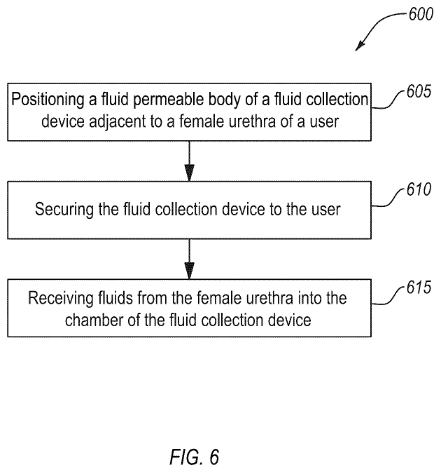

FIG. 6 is a flow diagram of a method 600 for collecting fluids. The method 600 includes an act 605 of positioning a fluid permeable body of a fluid collection device adjacent to a female urethra of a user. The fluid permeable body is disposed within a chamber of a fluid impermeable barrier of the fluid collection device and exposed to the female urethra of the user through an opening in the fluid collection device defined by the fluid impermeable barrier. The method 600 also includes an act 610 of securing the fluid collection device to the user. The method 600 also includes an act 615 of receiving fluids from the female urethra into the chamber of the fluid collection device. In some embodiments, the method 600 an act of applying suction effective to suction the fluids from the chamber via a conduit disposed therein.

Acts 605, 610, and 615 of the method 600 are for illustrative purposes. For example, the acts 605, 610, and 615 of the method 600 may be performed in different orders, split into multiple acts, modified, supplemented, or combined. In an embodiment, one or more of the acts 605, 610, and 615 of the method 600 may be omitted from the method 600. Any of the acts 605, 610, and 615 may include using any of the fluid collection devices or systems disclosed herein.

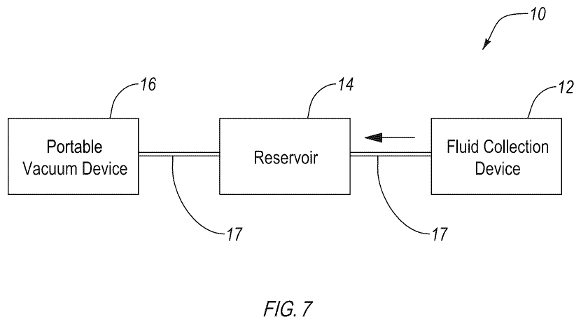

FIG. 7 is a block diagram of a system 10 for fluid collection, according to an embodiment. The system 10 includes a fluid collection device 12, a fluid storage container 14, and a portable vacuum source 16. The fluid collection device 12 may include any of the fluid collection devices described herein, such as the fluid collection device 100. The fluid collection device 12, the fluid storage container 14, and the portable vacuum source 16 may be fluidly coupled to each other via one or more conduits 17. The conduit 17 may include any of the conduits described herein, such as the conduit 108. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid collection device 12 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14.

The fluid collection device 12 may be shaped and sized to be positioned adjacent to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed:

1. A fluid collection device, comprising:
a fluid impermeable barrier having a proximal end, a proximal region including a first outer surface portion and defining an aperture extending therethrough, a distal end distal to the proximal end, a distal region including a second outer surface portion having more adhesive strength than the first outer surface portion of the proximal region, and a longitudinal length extending from the proximal end to the distal end, wherein the distal region extends less than about one-half of the longitudinal length such that the second outer surface portion that has more adhesive strength than the first outer surface portion terminates less than about one-half the longitudinal length from the distal end, the fluid impermeable barrier also defining an opening extending longitudinally along the fluid impermeable barrier and configured to be positioned adjacent to a female urethra and having an inner surface at least partially defining a chamber; and
a fluid permeable body positioned at least partially within the chamber to extend across at least a portion of the opening and configured to wick fluid away from the opening.

2. The fluid collection device of claim 1, wherein the second outer surface portion includes a lower surface roughness than the first outer surface portion.

3. The fluid collection device of 1, wherein the second outer surface portion terminates less than one-third of the longitudinal length from the distal end.

4. The fluid collection device of claim 1, wherein the proximal region and the distal region of the fluid impermeable barrier include at least one silicone or thermoplastic elastomer, and the distal region of the fluid impermeable barrier has a higher oil content that the proximal region.

5. The fluid collection device of claim 4, wherein at least the distal region of the fluid impermeable barrier includes about 60 oil parts per hundred rubber (phr) to about 200 oil phr based on 100 phr of styrene block co-polymer.

6. The fluid collection device of claim 4, wherein the second outer surface portion has more tack than the first outer surface portion.

7. The fluid collection device of claim 1, further comprising a conduit including an inlet and an outlet, wherein at least a portion of the conduit extends through the aperture in the proximal region and into the chamber, the fluid permeable body being configured to wick fluid away from the opening to the inlet.

8. The fluid collection device of claim 1, wherein the distal region includes an adhesive on the second outer surface portion.

9. The fluid collection device of claim 1, wherein the longitudinal length is at least about 20 cm.

10. A method of forming a fluid impermeable barrier of a fluid collection device, the method comprising:
forming a fluid impermeable barrier having a proximal end, a proximal region including a first outer surface portion and defining an aperture extending therethrough, a distal end distal to the proximal end, a distal region including a second outer surface portion having more adhesive strength than the first outer surface portion of the proximal region, and a longitudinal length extending from the proximal end to the distal end, wherein the distal region extends less than about one-half of the longitudinal length such that the second outer surface portion that has more adhesive strength than the first outer surface portion terminates less than about one-half the longitudinal length from the distal end, the fluid impermeable barrier also defining an opening extending longitudinally along the fluid impermeable barrier and configured to be positioned adjacent to a female urethra and having an inner surface at least partially defining a chamber; and
positioning a fluid permeable body at least partially within the chamber to extend across at least a portion of the opening and configured to wick fluid away from the opening.

11. The method of claim 10, wherein the second outer surface portion includes a lower surface roughness than the first outer surface portion.

12. The method of claim 10, wherein forming the fluid impermeable barrier includes:
inserting a composition including at least one of silicone or thermoplastic elastomer (TPE) into a barrier mold, the barrier mold including a first mold portion having a first mold surface and a second portion having a second mold surface more polished than the first mold surface; and
molding the composition in the barrier mold to form the fluid impermeable barrier with the first mold surface interfacing the first outer surface portion of the fluid impermeable barrier and the second mold surface interfacing the second outer surface portion of the fluid impermeable barrier.

13. The method of claim 12, further comprising mixing the at least one of the silicone or the TPE with oil to form the composition.

14. The method of claim 12, further comprising mixing a portion of the at least one of the silicone or the TPE with oil to form a second composition having a higher oil content than a first composition, wherein inserting a composition including at least one of silicone or TPE into the barrier mold includes:
inserting the first composition into the first mold portion of the barrier mold; and
inserting the second composition into the second mold portion of the barrier mold.

15. The method of claim 14, wherein the second composition includes about 60 oil parts per hundred rubber (phr) to about 200 oil phr based on 100 phr of styrene block co-polymer.

16. The method of claim 10, further comprising applying an adhesive to the second outer surface portion of the fluid impermeable barrier.

17. A fluid collection device, comprising:
a fluid impermeable barrier defining a chamber and an opening extending longitudinally along the fluid impermeable barrier and configured to be positioned adjacent to a female urethra, the fluid impermeable barrier including an outer surface having a first region and a second region having more adhesive strength than the first region of the outer surface, wherein at least the second region of the outer surface is at least partially curved to protrude outward and away from a longitudinal axis of the fluid impermeable barrier; and
a fluid permeable body positioned at least partially within the chamber to extend across at least a portion of the opening and configured to wick fluid away from the opening.

18. The fluid collection device of claim 17, wherein the second region includes a lower surface roughness than the first region of the outer surface of the fluid impermeable.

19. The fluid collection device of claim 17, wherein the second region has a higher oil content that the first region of the outer surface of the fluid impermeable.

20. The fluid collection device of claim 19, wherein the second region of the outer surface of the fluid impermeable barrier includes about 60 oil parts per hundred rubber (phr) to about 200 oil phr based on 100 phr of styrene block co-polymer.

21. The fluid collection device of claim 19, wherein the second region has more tack than the first region of the outer surface of the fluid impermeable.

22. The fluid collection device of claim 17, further comprising a conduit including an inlet and an outlet, wherein at least a portion of the conduit extends through an aperture in the proximal region and into the chamber, the fluid permeable body being configured to wick fluid away from the opening to the inlet.

23. The fluid collection device of claim 17, wherein the fluid impermeable barrier has a longitudinal length of at least about 20 cm.

24. A method of forming a fluid collection device, the method comprising:
forming a fluid impermeable barrier defining a chamber and an opening extending longitudinally along the fluid impermeable barrier and configured to be positioned adjacent to a female urethra, the fluid impermeable barrier including an outer surface having a first region and a second region having more adhesive strength than the first region of the outer surface, wherein at least the second region is at least partially curved to protrude outward and away from a longitudinal axis of the fluid impermeable barrier; and
positioning a fluid permeable body at least partially within the chamber to extend across at least a portion of the opening and configured to wick fluid away from the opening.

25. The method of claim 24, wherein the second region includes a lower surface roughness than the first region of the outer surface of the fluid impermeable.

26. The method of claim 24, wherein the second region has a higher oil content that the first region of the outer surface of the fluid impermeable.

27. The method of claim 26, wherein the second region of the outer surface of the fluid impermeable barrier includes about 60 oil parts per hundred rubber (phr) to about 200 oil phr based on 100 phr of styrene block co-polymer.

28. The method of claim 26, wherein the second region has more tack than the first region of the outer surface of the fluid impermeable.
